(19) United States
(12) Patent Application Publication
(10) Pub. No.: US 2022/0218649 A1
(43) Pub. Date: Jul. 14, 2022

(54) COMPOSITION FOR TREATING OPIOID WITHDRAWAL AND METHOD OF MANUFACTURE

(71) Applicant: Mai Nguyen, Salt Lake City, UT (US)

(72) Inventor: Mai Nguyen, Salt Lake City, UT (US)

(21) Appl. No.: 17/143,553

(22) Filed: Jan. 7, 2021

(51) Int. Cl. A61K 31/352; A61K 31/05; A61K 9/48; A61K 31/015; A61K 36/07; A61K 36/9066; A61K 36/752; A61K 36/67; A61K 36/258; A61K 36/53

(52) U.S. Cl. CPC A61K 31/352; A61K 31/05; A61K 9/4891; A61K 9/4833; A61K 9/4808; A61K 36/53; A61K 36/07; A61K 36/9066; A61K 36/752; A61K 36/67; A61K 36/258; A61K 31/015

(57) ABSTRACT

A composition for treating opioid withdrawal and method of manufacture and administration of the composition creates a synergistic combination of cannabinoids, terpenoids, herbs, and medicinal mushrooms that help to eliminate the symptoms of opioid withdrawal by creating a medicinal entourage. The composition combines: between about 2.5 to 100 milligrams of a cannabinoid, between about 0.084 to 33.6 milligrams of a terpenoid, between about 0.5 to 3000 milligrams of an herb, and between about 25 to 2500 milligrams of a medicinal mushroom. The composition is orally administered in a lipid-enclosed capsule form. The terpenoid has a lipophilic nature that enhances the bioavailability of the cannabinoid. The herb works with the body's endocannabinoid system to enhance pain relief capacity of the cannabinoid, so as to help inhibit symptoms of opioid withdrawal. The medicinal mushroom comprises a psychedelic drug that creates a therapeutic effect with the cannabinoid to inhibit symptoms of opioid withdrawal.

### FIELD OF THE INVENTION

This invention relates to a composition for treating opioid withdrawal and method of manufacture and more particularly relates to a composition consisting of naturally sourced extracts of cannabinoids, terpenoids, herbs, and medicinal mushrooms formulated to alleviate the symptoms of heroin and opioid withdrawal; whereby the ingredients create a medicinal entourage effect with the cannabinoid to enhance the bioavailability of the cannabinoid, and collectively thereby remediating and partially inhibiting symptoms of opioid withdrawal; whereby the ingredients are varied to produce both daytime and nighttime embodiments, and a first withdrawal phase and a second withdrawal phase version of the composition.

### BACKGROUND

### Description of the Related Art

Typically, heroin is an illegal, highly addictive drug processed from morphine, a naturally occurring substance extracted from the seed pod of certain varieties of poppy plants. Heroin binds to and activates specific receptors in the brain called mu-opioid receptors (MORs). Our bodies contain naturally occurring chemicals called neurotransmitters that bind to these receptors throughout the brain and body to regulate pain, hormone release, and feelings of well-being. When MORs are activated in the reward center of the brain, they stimulate the release of the neurotransmitter dopamine, causing a reinforcement of drug taking behavior.

Once heroin enters the brain, it is converted to morphine and binds rapidly to opioid receptors. People who use heroin typically report feeling a surge of pleasurable sensation—a “rush” or “high.” Repeated heroin use changes the physical structure and physiology of the brain, creating long-term imbalances in neuronal and hormonal systems which are not easily reversed. With physical dependence, the body adapts to the presence of the drug, and withdrawal symptoms occur if use is reduced abruptly. Withdrawal may occur within a few hours after the last use of the drug. Symptoms of withdrawal include restlessness, muscle and bone pain, insomnia, diarrhea, vomiting, cold flashes with goose bumps (“cold turkey”), and leg movements. With the physical dependence likewise comes psychological dependence, which may also manifest itself physiologically.

Opioids are a class of drugs that includes not just heroin, but also synthetic opioids such as fentanyl and pain relievers available legally by prescription, such as oxycodone (OxyContin®), hydrocodone (Vicodin®), codeine, morphine, and many others. Opioid receptors may be found not just in the brain, but also the spinal cord and gastrointestinal tract. Over time, an addicts body needs more and more of the drug to achieve the same effect and the receptors become desensitized. The tendency of addicts to ramp up dosages of the opioids can be very dangerous and increase the risk of accidental overdose. Prolonged use of these drugs changes neural pathways and the way nerve receptors function within the brain. The receptors become tolerant of the drug and eventually dependent upon the drug to function normally.

The withdrawal symptoms experienced depend on the phase of withdrawal being experienced. Multiple factors dictate how long a person will experience the symptoms of withdrawal. Early symptoms that occur in in the first withdrawal phase typically begin in the first 24 hours after the drug user stop using the opioid. The first withdrawal phase symptoms may include: muscle aches, restlessness, anxiety, lacrimation, runny nose, and inability to sleep. Later symptoms, which can be more intense, begin after the first day or so, include: diarrhea, abdominal cramping, goose bumps on skin, dilated pupils, and high blood pressure.

Opioid drug users with an opioid use disorder are often treated with opioid replacement therapy using methadone or buprenorphine. Being on such treatment reduces the risk of death. Additionally, cognitive behavioral therapy and other forms of support from mental health professionals such as individual or group therapy and peer support programs are utilized to help in withdrawal. It is also known in the art that Naloxone is useful for treating an opioid overdose and preventing relapse. Further, medical cannabis is used for treating and alleviating symptoms such as pain, anorexia, asthma, glaucoma, arthritis, spasms, anxiety, and substance withdrawal.

Other proposals have involved chemical compositions and methods for treating opioid and heroin withdrawals. The problem with these compositions is that they do not create a medicinal entourage effect with the cannabinoid to inhibit symptoms of opioid withdrawal. Also, the composition does not utilize a terpenoid, an herb, and a medicinal mushroom with the cannabinoid. Even though the above cited chemical compositions and methods for treating opioid and heroin withdrawals meet some of the needs of the market, a composition for treating opioid withdrawal and method of manufacture and more particularly relates to a synergistic combination consisting of naturally sourced extracts of cannabinoids, terpenoids, herbs, and medicinal mushrooms that help to eliminate the symptoms of heroin and opioid withdrawal, is still desired.

### SUMMARY

From the foregoing discussion, it should be apparent that a need exists for a composition for treating opioid withdrawal and method of manufacture and administration of the composition. Beneficially, such a composition would create a synergistic combination of cannabinoids, terpenoids, herbs, and medicinal mushrooms that help to eliminate the symptoms of opioid withdrawal. The ingredients can be varied in concentration and quantity to produce both, a daytime and a nighttime version, and a first withdrawal phase and a second withdrawal phase version of the composition.

The present invention has been developed in response to the present state of the art, and in particular, in response to the problems and needs in the art that have not yet been fully solved by currently available methods of treating heroin and opioid addiction using cannabinoid-related compositions.

Accordingly, the present invention has been developed to provide a composition in the form of an orally administered lipid-based capsule that overcome many or all of the above-discussed shortcomings in the art.

The composition for treating opioid withdrawal comprises: between about 2.5 to 100 milligrams of a cannabinoid, between about 0.084 to 33.6 milligrams of a terpenoid, between about 0.5 to 3000 milligrams of an herb, and between about 25 to 2500 milligrams of a medicinal mushroom.

Whereby the lipophilic nature of the terpenoid creates a medicinal entourage effect with the cannabinoid to enhance the bioavailability of the cannabinoid, so as to help inhibit symptoms of opioid withdrawal.

Whereby the herb binds to and activates cannabinoid receptors in the body to enhance the pain relief capacity of the cannabinoid, so as to help inhibit symptoms of opioid withdrawal.

Whereby the medicinal mushroom comprises a psychedelic compound that produce a therapeutic effect to enhance the pain relief capacity of the cannabinoid, so as to help inhibit symptoms of opioid withdrawal

Whereby the terpenoid consists of a terpene molecule and an alcohol molecule that create a medicinal entourage effect with the cannabinoid to help inhibit symptoms of opioid withdrawal. Whereby the herb works in conjunction with the body's endocannabinoid system to bind to and activate cannabinoid receptors to enhance the pain relief capacity of the cannabinoid, so as to help inhibit symptoms of opioid withdrawal. Whereby the medicinal mushroom comprises a triterpene and a psychedelic drug that create a medicinal entourage effect with the cannabinoid to help inhibit symptoms of opioid withdrawal.

In another embodiment, the cannabinoid is selected from the group consisting of: cannabinol (CBN), cannabidiol (CBD), cannabigerol (CBG), cannabichromene (CBC), and Delta-9 ttetrahydrocannabinol (THC), and mixtures thereof.

In another embodiment, the terpenoid is selected from the group consisting of: a-Pinene, Mycerne, b-Caryophyllene, a-Bisabolol, Linalool, Limonene, Terpinolene, Eucalyptol, Terpineol, and mixtures thereof.

In another embodiment, the terpenoid is between about 0.084 to 33.6 milligrams of the composition, or between about 0.3% to 0.6% of the composition.

In another embodiment, the herb is selected from the group consisting of: Tumeric, Cucumin, Bromelain, Pineapple, Capsaicin, Devil's Claw, Harpagoside, Ginseng, Ginsenosides, and mixtures thereof.

In another embodiment, the medicinal mushroom is selected from the group consisting of: Lion's Mane, Hericium erinaceus, Reishi, Ganoderma lucidum, Cordyceps, Turkey Tail, Trametes versicolor, Shiitake, Maitake, and mixtures thereof.

In another embodiment, the medicinal mushroom comprises a Triterpene, a Psychedelic Drug, a Polysaccharide, a polysaccharide-protein and polysaccharide-peptide complex, a Ribonucleases, a Proteases, a Lectin, a Lactone, an Alkaloid, an antioxidant, a flavonoid, and a phenol that create a medicinal entourage effect with the cannabinoid to help inhibit symptoms of opioid withdrawal.

In yet another embodiment, the composition is composed in with a first set of ingredients for a daytime embodiment, and a second set of ingredients for a nighttime embodiment.

In yet another embodiment, the daytime embodiment of the composition induces an energizing and anti-anxiety effect.

In yet another embodiment, the nighttime embodiment of the composition induces a seducing effect.

In yet another embodiment, the daytime embodiment of the composition comprises about: 2.5 to 100 milligrams of the Delta-9 ttetrahydrocannabinol (THC), 2.5 to 100 milligrams of the cannabidiol (CBD), 2.5 to 100 milligrams of the cannabigerol (CBG), 0.084 to 33.6 milligrams of the a-Pinene, 0.084 to 33.6 milligrams of the Mycerne, 0.084 to 33.6 milligrams of the b-Caryophyllene, 0.084 to 33.6 milligrams of the a-Bisabolol, 0.084 to 33.6 milligrams of the Linalool, 0.084 to 33.6 milligrams of the Limonene, 0.084 to 33.6 milligrams of the Terpinolene, 0.084 to 33.6 milligrams of the Eucalyptol, and 0.084 to 33.6 milligrams of the Terpineol, 100 to 2000 milligrams of the Tumeric, 100 to 2000 milligrams of the Cucumin, 10 to 400 milligrams of the Bromelain, 10 to 400 milligrams of the Pineapple, 0.5 to 6 milligrams of the Capsaicin, 50 to 1000 milligrams of the Devil's Claw, 50 to 1000 milligrams of the Harpagoside, 25 to 400 milligrams of the Ginseng, 25 to 400 milligrams of the Ginsenosides, 25-3000 milligrams of the Lion's Mane, 25-3000 milligrams of the Hericium erinaceus, 25-3000 milligrams of the Reishi, 25-3000 milligrams of the Ganoderma lucidum, 25-3000 milligrams of the Cordyceps, 25-3000 milligrams of the Turkey Tail, 25-3000 milligrams of the Trametes versicolor, 25-1000 milligrams of the Shiitake, and 25-2500 milligrams of the Maitake.

In yet another embodiment, the nighttime embodiment of the composition comprises about: 2.5 to 100 milligrams of the cannabinol (CBN), 2.5 to 100 milligrams of the Delta-9 ttetrahydrocannabinol (THC), 2.5 to 100 milligrams of the cannabidiol (CBD), 2.5 to 100 milligrams of the cannabigerol (CBG), 0.084 to 33.6 milligrams of the a-Pinene, 0.084 to 33.6 milligrams of the Mycerne, 0.084 to 33.6 milligrams of the b-Caryophyllene, 0.084 to 33.6 milligrams of the a-Bisabolol, 0.084 to 33.6 milligrams of the Linalool, 0.084 to 33.6 milligrams of the Limonene, 0.084 to 33.6 milligrams of the Terpinolene, 0.084 to 33.6 milligrams of the Eucalyptol, and 0.084 to 33.6 milligrams of the Terpineol, 100 to 2000 milligrams of the Tumeric, 100 to 2000 milligrams of the Cucumin, 10 to 400 milligrams of the Bromelain, 10 to 400 milligrams of the Pineapple, 0.5 to 6 milligrams of the Capsaicin, 50 to 1000 milligrams of the Devil's Claw, 50 to 1000 milligrams of the Harpagoside, 25 to 400 milligrams of the Ginseng, 25 to 400 milligrams of the Ginsenosides, 25-3000 milligrams of the Lion's Mane, 25-3000 milligrams of the Hericium erinaceus, 25-3000 milligrams of the Reishi, 25-3000 milligrams of the Ganoderma lucidum, 25-3000 milligrams of the Cordyceps, 25-3000 milligrams of the Turkey Tail, 25-3000 milligrams of the Trametes versicolor, 25-1000 milligrams of the Shiitake, and 25-2500 milligrams of the Maitake.

In yet another embodiment, the composition comprises a first set of ingredients for a first withdrawal phase embodiment.

In yet another embodiment, the composition comprises a second set of ingredients for a second withdrawal phase embodiment.

In yet another embodiment, the composition comprises a set of ingredients for a pain management embodiment.

In yet another embodiment, the composition is operable to help treat heroin withdrawal, synthetic opioid withdrawal, fentanyl withdrawal, oxycodone withdrawal, hydrocodone withdrawal, codeine withdrawal, and morphine withdrawal.

In yet another embodiment, the composition is administered through a lipid-based capsule.

A method of the present invention is also presented for manufacturing the composition. The method in the disclosed embodiments substantially includes the steps necessary to carry out the functions presented above with respect to the operation of the described apparatus and system. In one embodiment, the method includes a Step of soaking a cannabinoid, a terpenoid, an herb, and a medicinal mushroom in a liquid. The method also may include a Step of combining the cannabinoid, the terpenoid, the herb, and the medicinal mushroom.

In a further embodiment, the method includes a Step of decocting the combination of the cannabinoid, the terpenoid, the herb, and the medicinal mushroom. Another Step comprises combining, distilling, and evaporating the concentrated liquids to produce a dry powder. Yet another Step may include forming the dry powder into a capsule. A Step comprises encapsulating the capsule into a lipid-based panel. A final Step comprises orally administering the lipid coated capsule.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a formulation Table that lists the ingredients and quantities thereof necessary to manufacture a composition for treating the first withdrawal phase of opioid withdrawal, in accordance with an embodiment of the present invention;

FIG. 2 is a formulation Table that lists the ingredients and quantities thereof necessary to manufacture a composition for treating the second withdrawal phase of opioid withdrawal, in accordance with an embodiment of the present invention;

FIG. 3 is a formulation Table that lists the ingredients and quantities thereof necessary to manufacture a composition for managing pain and symptoms during opioid withdrawal, in accordance with an embodiment of the present invention;

FIG. 4 is a Table that lists the different types of cannabinoids, in accordance with an embodiment of the present invention;

FIG. 5 is a Table that lists the different types of terpenoids, in accordance with an embodiment of the present invention;

FIG. 6 is a Table that lists the different types of herbs, in accordance with an embodiment of the present invention;

FIG. 7 is a Table that lists the different types of medicinal mushrooms, in accordance with an embodiment of the present invention; and

FIG. 8 is a flowchart referencing an exemplary method for manufacturing a composition for treating opioid withdrawal, in accordance with an embodiment of the present invention.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

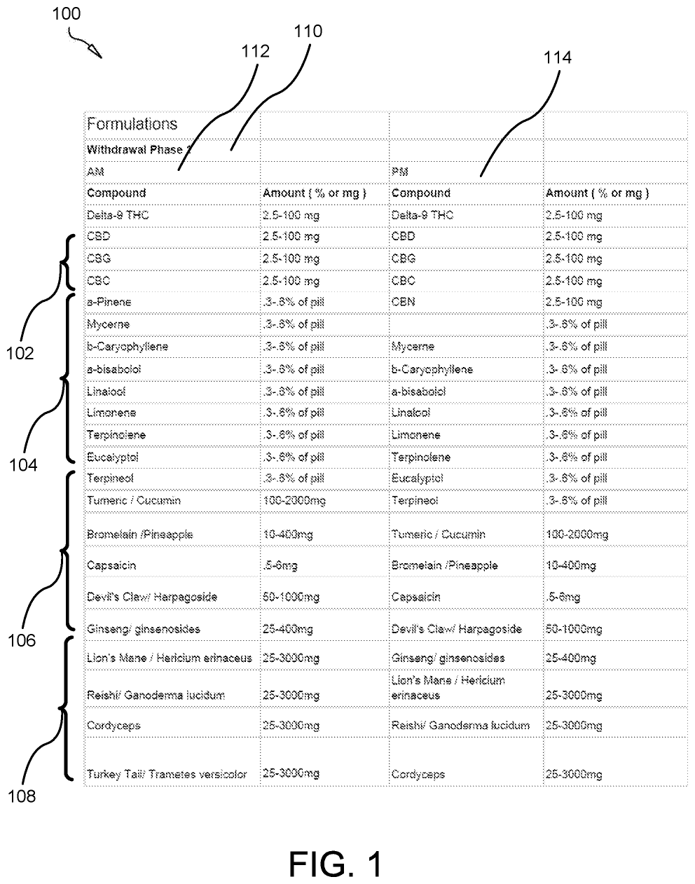

FIG. 1 references a formulation Table 100 that lists the ingredients and quantities thereof necessary to manufacture a composition 100 for treating the first withdrawal phase of opioid withdrawal. The composition 100 is a unique combination of ingredients that work together in a synergistic manner to alleviate symptoms associated withdrawal of opioids. The composition 100 provides a synergistic combination of cannabinoids 102, terpenoids 104, herbs 106, and medicinal mushrooms 108 that produce a medicinal entourage effect. The synergy between the ingredients, consequently helps eliminate the symptoms of opioid withdrawal.

Further, the composition 100 is efficacious for treating heroin withdrawal, synthetic opioid withdrawal, fentanyl withdrawal, oxycodone withdrawal, hydrocodone withdrawal, codeine withdrawal, and morphine withdrawal. Significantly, heroin, which is a type of opioid, is treated in substantially the same manner with substantially the same ingredients for the composition 100, as taught for treating and relieving the opioid withdrawal.

In one embodiment, a first ingredient utilized in the composition 100 is a cannabinoid 102, or derivatives thereof. The cannabinoid 102 activates cannabinoid receptors in the endocannabinoid system of the body. Another ingredient is a terpenoid 104 that creates an entourage effect by improving the bioavailability of the cannabinoid 102. Yet another ingredient is an herb 106 that serves to increase binding by the cannabinoid 102 to the cannabinoid receptors, which further enhances pain relief Another ingredient used in the composition 100 is a medicinal mushroom 108. The mushroom 108, and variants thereof, is characterized with psychedelic compounds that produce a therapeutic effect, which enhances the pain relief capacity of the cannabinoid 102. These ingredients can have different variations and quantities for use in the composition 100.

Additionally, the ingredients can be varied in concentration and quantity to produce both, a daytime version 112 of the composition, and a slightly different nighttime version 114 of the composition. The daytime version 112 provides more energy to the user, while the nighttime version helps the sedate the user for facilitated sleeping. Both daytime version and nighttime version 112, 114 of the composition 100 are shown in Table 100 and Table 200.

Additionally, the ingredients can be altered slightly to accommodate the multiple withdrawal phases of opioid withdrawal. For example, first and second withdrawal phase embodiments 110, 202 of the composition 100 are manufactured through a slight alteration of ingredients and quantities thereof. Table 100 shows the first withdrawal phase embodiment 110, and Table 200 shows the first withdrawal phase embodiment 202.

A significant aspect of the present disclosure is the medical entourage effect, and synergy created between the primary ingredients, i.e., cannabinoid 102, terpenoid 104, herb 106, and medicinal mushroom 108. For example, it is known in the art that terpenoids are natural volatile non-aromatic compounds found as components of essential oils present in many plants and contain a carbon, hydrogen, and oxygen scaffold. Terpenoids have been used as fragrances and flavoring agents, as well as skin penetration agents. Terpenoids have poor solubility in water, but are readily soluble in non-aqueous and/or hydrophobic medium. Because of their lipophilic nature, terpenoids can easily cross the blood-brain barrier and interact with cell membranes by binding to membrane receptors.

Cannabinoids are compounds derived from Cannabis sativa, an annual plant in the Cannabaceae family. The plant contains about 60 cannabinoids. The most active naturally occurring cannabinoid 102 is tetrahydrocannabinol (THC), which is used for the treatment of a wide range of medical conditions, including glaucoma, AIDS wasting, neuropathic pain, treatment of spasticity associated with multiple sclerosis, fibromyalgia and chemotherapy-induced nausea. Additionally, THC has been reported to exhibit a therapeutic effect in the treatment of allergies, inflammation, infection, epilepsy, depression, migraine, bipolar disorders, anxiety disorder, and drug dependency and withdrawal syndromes. THC is particularly effective as an anti-emetic drug and is administered to curb emesis, a common side effect accompanying the use of opioid analgesics and anaesthetics, highly active anti-retroviral therapy and cancer chemotherapy.

Like terpenes, cannabinoids are lipophilic and potentially acid-labile compounds. Because of their hydrophobic nature, cannabinoids are poorly absorbed systemically from oral dosage forms in the aqueous environment of the gastrointestinal tract, and oral formulations of cannabinoids, therefore, exhibit low bioavailability.

Those skilled in the art of pharmacology will recognize that bioavailability is a subcategory of absorption and is the fraction (%) of an administered drug that reaches the systemic circulation. However, because of the lipophilic nature of terpenoids, the terpenoid 104 can bind to the cannabinoid 102, which enhances the bioavailability of the cannabinoid 102. For example, bioavailability enhances the capacity to cross the blood-brain barrier and interact with cell membranes by binding to membrane receptors.

Similarly, the molecular configuration of the herb 106 creates a medical entourage with the cannabinoid 102. The herb 106 has inherent characteristics that increase binding by the cannabinoid 102 to the cannabinoid receptors. This, in turn, alleviates pain during the opioid withdrawal. However, since multiple types of herbs may be used, the efficacy can vary.

Another medical entourage is provided by the medicinal mushroom 108, which produces a therapeutic effect to enhance the pain relief capacity of the cannabinoid 102. The combined effect of the psychedelic compound in the mushroom 108, and the cannabinoid 102 work together to produce a therapeutic effect for alleviating the opioid withdrawal symptoms. Psychedelic compounds, which are inherent in such mushrooms have demonstrated therapeutic effect in a wide variety of clinical applications, including depression, anxiety, substance abuse, and a variety of other conditions. In addition, psychedelic compounds have also demonstrated therapeutic effect in inflammation related disease, at both perceptual and sub-perceptual dose levels

Looking again at Table 100, the composition 100 comprises a first set of ingredients that are designed for optimal efficacy in a first withdrawal phase embodiment 110. Early symptoms that occur in in the first withdrawal phase typically begin in the first 24 hours after the drug user stop using the opioid drug. Often, the first withdrawal phase symptoms include: muscle aches, restlessness, anxiety, lacrimation, runny nose, and inability to sleep. Later symptoms, which can be more intense, begin after the first day or so, include: diarrhea, abdominal cramping, goose bumps on skin, dilated pupils, and high blood pressure.

The ingredients for the first withdrawal phase embodiment 110 may include, without limitation, between about 2.5 to 100 milligrams of a cannabinoid 102. The lower end of 2.5 mg of cannabinoid 102 can be used for smaller doses and concentrations of composition 100, while the larger quantity of 100 mg can be used for more concentrated version of the composition 100, or for creating more dosages. In regards to the terpenoid 104, between 0.084 to 33.6 milligrams of the terpenoid 104 is added with the cannabinoid 102. In another embodiment, the terpenoid 104 is measured in weight percentage, containing between about 0.3% to 0.6% of the total composition 100.

Continuing with the first withdrawal phase embodiment 110 of the composition 100, the herb 106, which can contain an eclectic assortment herbs, is introduced into the composition 100 at between 0.5 to 3000 milligrams of an herb 106. The smaller quantity of 0.5 mg is used for smaller concentrations, and the larger quantity of 3000 mg is used for larger dosages. Finally, the medicinal mushroom 108 is added about 25 to 2500 milligrams, where 25 mg is used for less concentrated dosages, and 2500 mg are used for more concentrated or larger quantities of the composition 100.

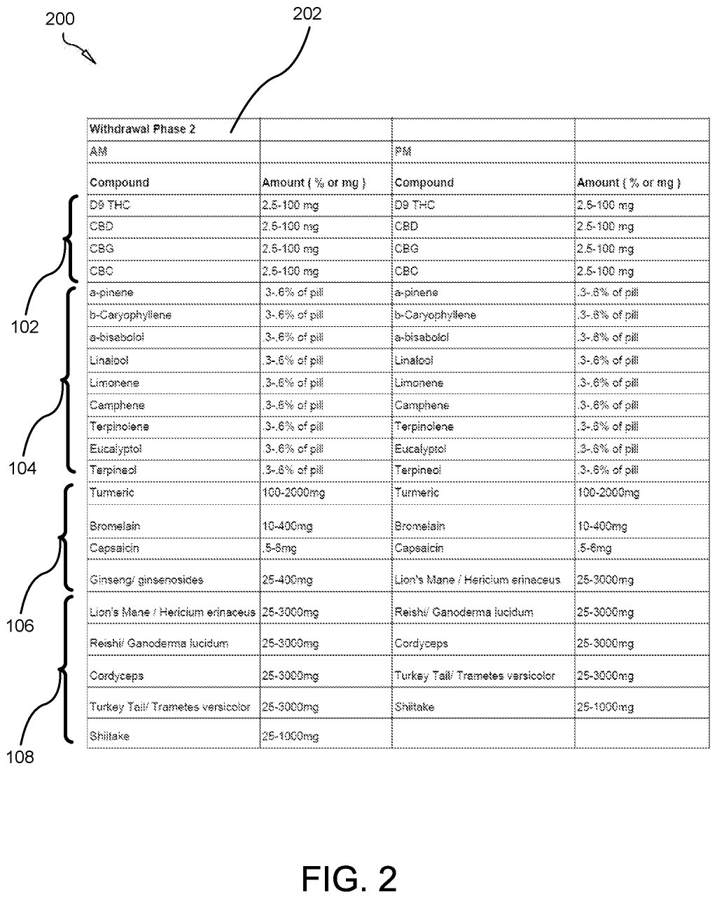

FIG. 2 references Table 200, which lists the ingredients for a second withdrawal phase embodiment 202, used during the second phase of opioid withdrawal. Often during the second withdrawal phase, nausea and vomiting are symptoms. Furthermore, inadvertent breathing of vomited material into the lungs (known as aspiration) can be a serious complication associated with the second withdrawal phase, as it can lead to the development of pneumonia. The ingredients used in the second withdrawal phase embodiment 202 are substantially the same in quantity, except that the Maitake mushroom 108 is excluded for the second withdrawal phase of opioid withdrawal. Further, the terpenoid 104, Camphene is excluded from the first withdrawal phase of opioid withdrawal.

The quantities of the composition 100 are manufactured through a unique process described below. The composition 100 is produced in capsule form, which has a lipid outer case that helps bioavailability and swallowing of the capsule. In one embodiment, the composition 100 is administered through a lipid-based capsule. However, in other embodiments, the composition 100 can be administered in gel form, liquid form, or injected intravenously.

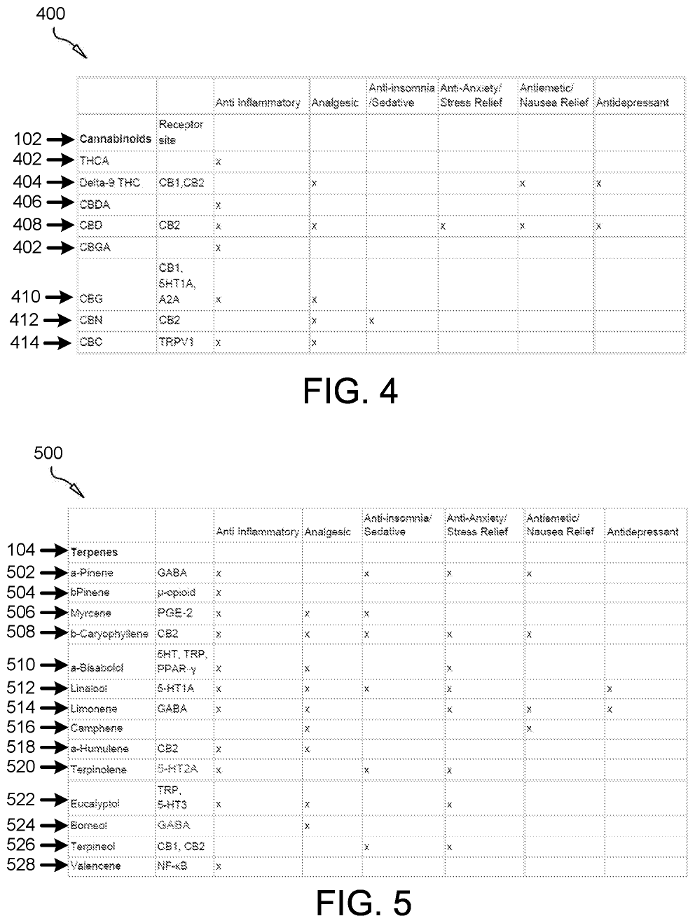

Looking first at the cannabinoid 102 in Table 400 (See FIG. 4), it is known in the art that medical cannabis is used for treating and alleviating symptoms such as pain, anorexia, asthma, glaucoma, arthritis, spasms, anxiety, and substance withdrawal. There is nascent evidence that suggests that cannabinoid 102 is an efficacious tool during the acute opioid withdrawal period. Numerous pre-clinical studies have shown that cannabis and cannabinoids decrease opioid withdrawal symptoms.

It is also known that a cannabinoid 102, and the THC compound thereof, binds and attaches to the cannabinoid receptors located in the endocannabinoid system of the body. This attachment serves to activate the cannabinoid receptors, which helps alleviate pain, and in some cases, the painful withdrawal effects of opioid symptoms.

Table 400 references that the cannabinoid 102 may include Cannabinol (CBN) 412, cannabidiol (CBD) 408, cannabigerol (CBG) 410, cannabichromene (CBC) 414, Delta-9 tetrahydrocannabinol (THC) 404, THCA 402, CBDA 406, and mixtures thereof. In some embodiments, the Delta-9 tetrahydrocannabinol 404 is efficacious for treating pain, muscle spasticity, glaucoma, insomnia, low appetite, nausea, and anxiety. The cannabidiol 408 is efficacious for treating anxiety, depression, seizures, inflammation, pain, ibs, nausea, and migraines. The CBDA 406 is efficacious for treating depression, epilepsy, breast cancer, anti-inflammatory. CBDA 406 can be processed raw, or juiced. The cannabigerol 410 is used as an anti-inflammatory and pain reliever. These are all symptoms of opioid withdrawal.

Turning now to FIG. 5, the terpenoid 104, and variations thereof, is referenced. The terpenoid 104 is utilized in the composition 100 to produce a medicinal entourage effect with the cannabinoid 102 that helps eliminate the symptoms of opioid withdrawal. The lipophilic nature of the terpenoid 104 creates a medicinal entourage effect with the cannabinoid 102 to enhance the bioavailability of the cannabinoid 102, so as to help inhibit symptoms of opioid withdrawal. In another embodiment, the terpenoid 104 consists of a terpene molecule and an alcohol molecule that create a medicinal entourage effect with the cannabinoid 102 to help inhibit symptoms of opioid withdrawal.

Table 500 shows that the terpenoid 104 may include, without limitation, a-Pinene 502, Mycerne 506, b-Caryophyllene 508, a-Bisabolol 510, Linalool 512, Limonene 514, Terpinolene 520, Eucalyptol 522, Terpineol 526, Camphene 516, a-Humulene 518, Borneol 524, Valencene 528, and mixtures thereof. The a-Pinene 502 is efficacious for treating pain, anxiety, and inflammation. The Limonene 514 is efficacious for treating inflammation, pain, stress, and anxiety. The Myrcene 506 is efficacious for treating pain, anxiety, muscle relaxant, and use as a sedative. The Borneol 524 helps with chronic and acute pain. The Eucalyptol 522 serves as an anti-inflammatory to reduce pain. These are all symptoms of opioid withdrawal.

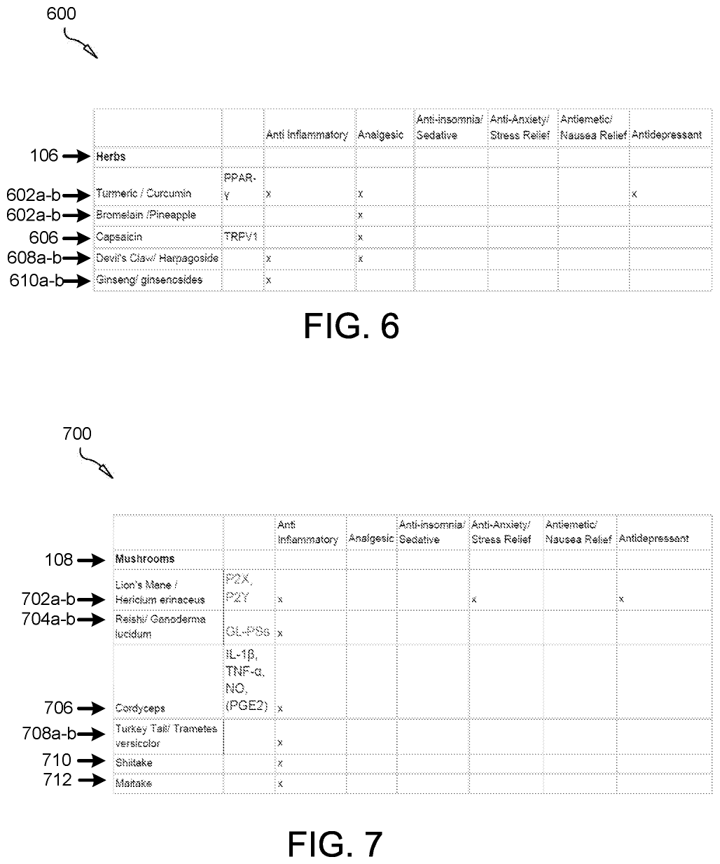

As referenced in FIG. 6, an herb 106 may also be used in the composition 100. The inherent characteristics of the herb 106 work with the cannabinoid 102 to help inhibit symptoms of opioid withdrawal. The herb 106 is efficacious in binding cannabinoid receptors in the body's endocannabinoid system, which enhances the pain relief capacity of the cannabinoid 102. As with the terpenoid 104, this medical entourage with the cannabinoid 102 helps inhibit symptoms of opioid withdrawal.

Looking at Table 600, the herb 106 may include, without limitation, Tumeric 602a, Curcumin 602b, Bromelain 604a, Pineapple 604b, Capsaicin 606, Devil's Claw 608a, Harpagoside 608b, Ginseng 610a, Ginsenosides 610b, and mixtures thereof. The Bromelain 604a is efficacious for treating muscle spasms and pain. The Capsaicin 606 helps with pain relief. The Devil's Claw 608a helps relieve fever and arthritis. These are all symptoms of opioid withdrawal.

Finally, the medicinal mushroom 108 is used in the compound (See Table 700). It is known in the art that the medicinal mushroom 108 comprises a triterpene and a psychedelic drug that to help inhibit symptoms of opioid withdrawal. The psychedelic compound produces a therapeutic effect that enhances the pain relief capacity of the cannabinoid 102, which creates a medicinal entourage effect with the cannabinoid 102. Similar to the terpenoid 104, the mushroom 108 enhances the cannabinoid 102, so as to help inhibit symptoms of opioid withdrawal.

It is known in the art that the medicinal mushroom 108 may be characterized by having at least one of: Triterpene, a Psychedelic Drug, a Polysaccharide, a polysaccharide-protein and polysaccharide-peptide complex, a Ribonucleases, a Proteases, a Lectin, a Lactone, an Alkaloid, an antioxidant, a flavonoid, and a phenol. These compounds serve to create a medicinal entourage effect with the cannabinoid 102.

As shown in the Table 700 in FIG. 7, the medicinal mushroom 108, may include, without limitation, Lion's Mane 702a, Hericium erinaceus 702b, Reishi 704a, Ganoderma lucidum 704b, Cordyceps 706, Turkey Tail 708a, Trametes versicolor 708b, Shiitake 710, Maitake 712, and mixtures thereof. The Lions Mane 702a promotes healing nerve growth, and oxidative stress. The Cordyceps 706 increases physical performance. The Reishi 704a is efficacious for improving sleep, reducing stress, and fatigue. The shiake 710 helps with immunity, liver function, and supports the cardiovascular system. The Maitake 712 supports the immune system by stimulating immune systems killer cells and t-helper cells.

It is known in the art that such psychedelic compounds, which are inherent in mushrooms 108, have a demonstrated therapeutic effect in a wide variety of clinical applications, including depression, anxiety, substance abuse, and a variety of other conditions. In addition, psychedelic compounds have also demonstrated therapeutic effect in inflammation related disease, at both perceptual and sub-perceptual dose levels.

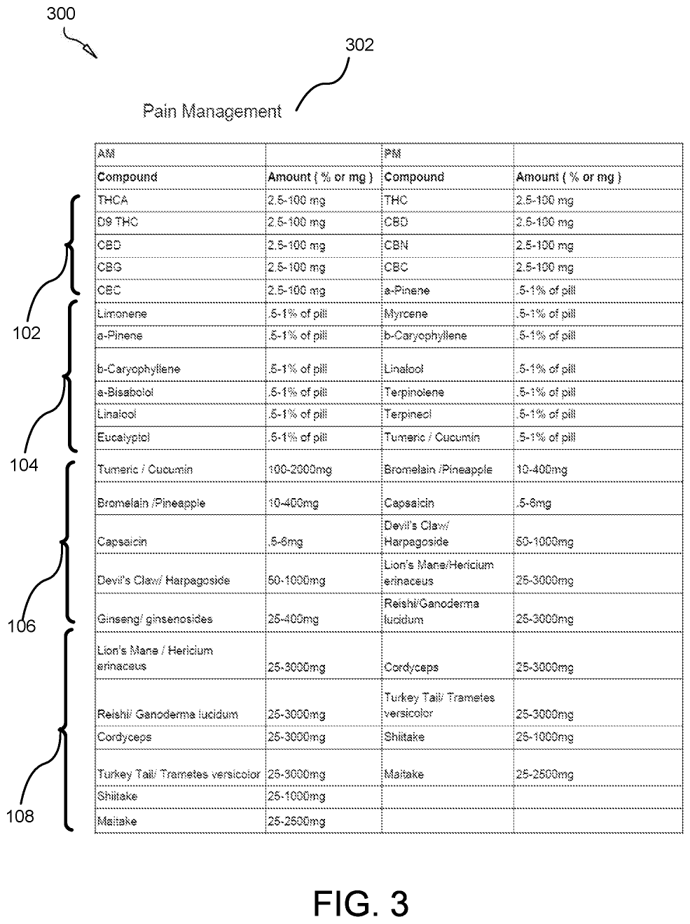

As discussed above, the composition 100 is composed in with a first set of ingredients for a daytime embodiment 112 for oral ingestion during daytime. A second set of ingredients for a nighttime embodiment 114 is efficacious for oral administration during the nighttime, when the user is likely to sleep. A third set of ingredients, shown in Table 300, are configured for a pain management embodiment 302 of the composition, used to alleviate pain during the withdrawal (FIG. 3). As referenced in Table 300, the pain management embodiment 302 has elevated quantities of the terpenoid 104, from 0.5% to 1% of the total composition 100. Otherwise, the ingredients and quantities thereof are substantially the same as the daytime version 112 of the composition 100.

The daytime embodiment 112 of the composition 100 induces an energizing and anti-anxiety effect. As shown in Table 1, the daytime embodiment 112 of the composition 100 comprises about: 2.5 to 100 milligrams of the Delta-9 tetrahydrocannabinol (THC), 2.5 to 100 milligrams of the cannabidiol (CBD), 2.5 to 100 milligrams of the cannabigerol (CBG), 0.084 to 33.6 milligrams of the a-Pinene, 0.084 to 33.6 milligrams of the Mycerne, 0.084 to 33.6 milligrams of the b-Caryophyllene, 0.084 to 33.6 milligrams of the a-Bisabolol, 0.084 to 33.6 milligrams of the Linalool, 0.084 to 33.6 milligrams of the Limonene, 0.084 to 33.6 milligrams of the Terpinolene, 0.084 to 33.6 milligrams of the Eucalyptol, and 0.084 to 33.6 milligrams of the Terpineol, 100 to 2000 milligrams of the Tumeric, 100 to 2000 milligrams of the Cucumin, 10 to 400 milligrams of the Bromelain, 10 to 400 milligrams of the Pineapple, 0.5 to 6 milligrams of the Capsaicin, 50 to 1000 milligrams of the Devil's Claw, 50 to 1000 milligrams of the Harpagoside, 25 to 400 milligrams of the Ginseng, 25 to 400 milligrams of the Ginsenosides, 25-3000 milligrams of the Lion's Mane, 25-3000 milligrams of the Hericium erinaceus, 25-3000 milligrams of the Reishi, 25-3000 milligrams of the Ganoderma lucidum, 25-3000 milligrams of the Cordyceps, 25-3000 milligrams of the Turkey Tail, 25-3000 milligrams of the Trametes versicolor, 25-1000 milligrams of the Shiitake, and 25-2500 milligrams of the Maitake.

The nighttime embodiment 114 of the composition induces a seducing effect. Looking again at FIG. 1, the nighttime embodiment 114 of the composition comprises about: 2.5 to 100 milligrams of the cannabinol (CBN), 2.5 to 100 milligrams of the Delta-9 tetrahydrocannabinol (THC), 2.5 to 100 milligrams of the cannabidiol (CBD), 2.5 to 100 milligrams of the cannabigerol (CBG), 0.084 to 33.6 milligrams of the a-Pinene, 0.084 to 33.6 milligrams of the Mycerne, 0.084 to 33.6 milligrams of the b-Caryophyllene, 0.084 to 33.6 milligrams of the a-Bisabolol, 0.084 to 33.6 milligrams of the Linalool, 0.084 to 33.6 milligrams of the Limonene, 0.084 to 33.6 milligrams of the Terpinolene, 0.084 to 33.6 milligrams of the Eucalyptol, and 0.084 to 33.6 milligrams of the Terpineol, 100 to 2000 milligrams of the Tumeric, 100 to 2000 milligrams of the Cucumin, 10 to 400 milligrams of the Bromelain, 10 to 400 milligrams of the Pineapple, 0.5 to 6 milligrams of the Capsaicin, 50 to 1000 milligrams of the Devil's Claw, 50 to 1000 milligrams of the Harpagoside, 25 to 400 milligrams of the Ginseng, 25 to 400 milligrams of the Ginsenosides, 25-3000 milligrams of the Lion's Mane, 25-3000 milligrams of the Hericium erinaceus, 25-3000 milligrams of the Reishi, 25-3000 milligrams of the Ganoderma lucidum, 25-3000 milligrams of the Cordyceps, 25-3000 milligrams of the Turkey Tail, 25-3000 milligrams of the Trametes versicolor, 25-1000 milligrams of the Shiitake, and 25-2500 milligrams of the Maitake.

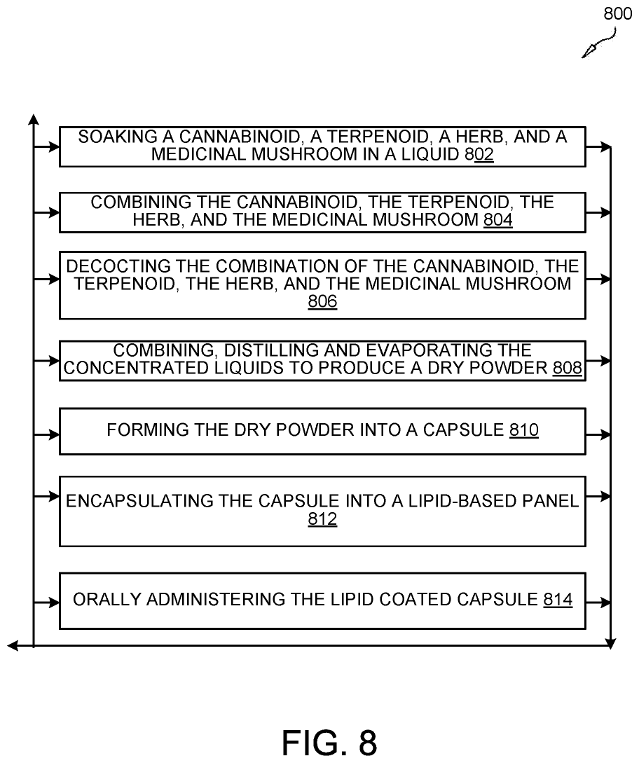

As discussed above, a method 800 of manufacturing the composition is also disclosed. FIG. 8 references a flowchart showing the steps for manufacture of the composition 100. The method 800 in the disclosed embodiments substantially includes the steps necessary to carry out the functions presented above with respect to the operation of the described apparatus and system. In one embodiment, the method 800 includes a Step 802 of soaking a cannabinoid, a terpenoid, an herb, and a medicinal mushroom in a liquid. These ingredients can be fresh, dried, or dehydrated. The ingredients can be in the form of herbal or plant material, which may include stems, roots, bark and rhizomes.

In some embodiments, the method 800 also may include a Step 804 of combining the cannabinoid, the terpenoid, the herb, and the medicinal mushroom. The ingredients can be combined in normal laboratory procedure under normal temperature and pressure. In a further embodiment, the method 800 includes a Step 806 of decocting the combination of the cannabinoid, the terpenoid, the herb, and the medicinal mushroom.

The decocting can include extraction by boiling the ingredients to dissolve the chemicals. Another Step 808 comprises combining, distilling, and evaporating the concentrated liquids to produce a dry powder. The combining, distilling, and evaporating is performed in normal laboratory procedure. Yet another Step 810 in the method 800 may include forming the dry powder into a capsule. Another Step 812 comprises encapsulating the capsule into a lipid-based panel. The lipid outer panel helps bioavailability and swallowing of the capsule. A final Step 814 comprises orally administering the lipid coated capsule.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A composition for treating opioid withdrawal, the composition comprising:
between about 2.5 to 100 milligrams of a cannabinoid;
between about 0.084 to 33.6 milligrams of a terpenoid;
between about 0.5 to 3000 milligrams of an herb; and
between about 25 to 2500 milligrams of a mushroom.

2. The composition of claim 1, wherein the lipophilic nature of the terpenoid creates a medicinal entourage effect with the cannabinoid to enhance the bioavailability of the cannabinoid, so as to help inhibit symptoms of opioid withdrawal.

3. The composition of claim 1, wherein the herb binds to and activates cannabinoid receptors in the body to enhance the pain relief capacity of the cannabinoid, so as to help inhibit symptoms of opioid withdrawal.

4. The composition of claim 1, wherein the mushroom comprises a psychedelic compound that produce a therapeutic effect to enhance the pain relief capacity of the cannabinoid, so as to help inhibit symptoms of opioid withdrawal.

5. The composition of claim 1, wherein the cannabinoid is selected from the group consisting of: cannabinol, cannabidiol, cannabigerol, cannabichromene, Delta-9 tetrahydrocannabinol, and mixtures thereof.

6. The composition of claim 1, wherein the terpenoid is selected from the group consisting of: a-Pinene, Mycerne, b-Caryophyllene, a-Bisabolol, Linalool, Limonene, Terpinolene, Eucalyptol, Terpineol, Camphene, and mixtures thereof.

7. The composition of claim 1, wherein the terpenoid is between about 0.084 to 33.6 milligrams of the composition, or between about 0.3% to 0.6% of the composition.

8. The composition of claim 1, wherein the herb is selected from the group consisting of: Tumeric, Curcumin, Bromelain, Pineapple, Capsaicin, Devil's Claw, Harpagoside, Ginseng, Ginsenosides, and mixtures thereof.

9. The composition of claim 1, wherein the mushroom is selected from the group consisting of: Lion's Mane, Hericium erinaceus, Reishi, Ganoderma lucidum, Cordyceps, Turkey Tail, Trametes versicolor, Shiitake, Maitake, and mixtures thereof.

10. The composition of claim 1, wherein the mushroom includes at least one of the following: a Triterpene, a Psychedelic Drug, a Polysaccharide, a polysaccharide-protein and polysaccharide-peptide complex, a Ribonucleases, a Proteases, a Lectin, a Lactone, an Alkaloid, an antioxidant, a flavonoid, and a phenol that create a medicinal entourage effect with the cannabinoid to help inhibit symptoms of opioid withdrawal.

11. The composition of claim 1, wherein the composition is composed in with a first set of ingredients for a daytime embodiment, and a second set of ingredients for a nighttime embodiment.

12. The composition of claim 11, wherein the daytime embodiment of the composition induces an energizing and anti-anxiety effect, and the nighttime embodiment of the composition induces a seducing effect.

13. The composition of claim 11, wherein the daytime embodiment of the composition comprises about: 2.5 to 100 milligrams of the Delta-9 tetrahydrocannabinol, 2.5 to 100 milligrams of the cannabidiol, 2.5 to 100 milligrams of the cannabigerol, 0.084 to 33.6 milligrams of the a-Pinene, 0.084 to 33.6 milligrams of the Mycerne, 0.084 to 33.6 milligrams of the b-Caryophyllene, 0.084 to 33.6 milligrams of the a-Bisabolol, 0.084 to 33.6 milligrams of the Linalool, 0.084 to 33.6 milligrams of the Limonene, 0.084 to 33.6 milligrams of the Terpinolene, 0.084 to 33.6 milligrams of the Eucalyptol, and 0.084 to 33.6 milligrams of the Terpineol, 100 to 2000 milligrams of the Tumeric, 100 to 2000 milligrams of the Cucumin, 10 to 400 milligrams of the Bromelain, 10 to 400 milligrams of the Pineapple, 0.5 to 6 milligrams of the Capsaicin, 50 to 1000 milligrams of the Devil's Claw, 50 to 1000 milligrams of the Harpagoside, 25 to 400 milligrams of the Ginseng, 25 to 400 milligrams of the Ginsenosides, 25-3000 milligrams of the Lion's Mane, 25-3000 milligrams of the Hericium erinaceus, 25-3000 milligrams of the Reishi, 25-3000 milligrams of the Ganoderma lucidum, 25-3000 milligrams of the Cordyceps, 25-3000 milligrams of the Turkey Tail, 25-3000 milligrams of the Trametes versicolor, 25-1000 milligrams of the Shiitake, and 25-2500 milligrams of the Maitake.

14. The composition of claim 11, wherein the nighttime embodiment of the composition comprises about: 2.5 to 100 milligrams of the cannabinol, 2.5 to 100 milligrams of the Delta-9 tetrahydrocannabinol, 2.5 to 100 milligrams of the cannabidiol, 2.5 to 100 milligrams of the cannabigerol, 0.084 to 33.6 milligrams of the a-Pinene, 0.084 to 33.6 milligrams of the Mycerne, 0.084 to 33.6 milligrams of the b-Caryophyllene, 0.084 to 33.6 milligrams of the a-Bisabolol, 0.084 to 33.6 milligrams of the Linalool, 0.084 to 33.6 milligrams of the Limonene, 0.084 to 33.6 milligrams of the Terpinolene, 0.084 to 33.6 milligrams of the Eucalyptol, and 0.084 to 33.6 milligrams of the Terpineol, 100 to 2000 milligrams of the Tumeric, 100 to 2000 milligrams of the Cucumin, 10 to 400 milligrams of the Bromelain, 10 to 400 milligrams of the Pineapple, 0.5 to 6 milligrams of the Capsaicin, 50 to 1000 milligrams of the Devil's Claw, 50 to 1000 milligrams of the Harpagoside, 25 to 400 milligrams of the Ginseng, 25 to 400 milligrams of the Ginsenosides, 25-3000 milligrams of the Lion's Mane, 25-3000 milligrams of the Hericium erinaceus, 25-3000 milligrams of the Reishi, 25-3000 milligrams of the Ganoderma lucidum, 25-3000 milligrams of the Cordyceps, 25-3000 milligrams of the Turkey Tail, 25-3000 milligrams of the Trametes versicolor, 25-1000 milligrams of the Shiitake, and 25-2500 milligrams of the Maitake.

15. The composition of claim 1, wherein the composition comprises a first set of ingredients for a first withdrawal phase embodiment, a second set of ingredients for a second withdrawal phase embodiment, and a third set of ingredients for a pain management embodiment.

16. The composition of claim 1, wherein the composition is operable to help treat heroin withdrawal, synthetic opioid withdrawal, fentanyl withdrawal, oxycodone withdrawal, hydrocodone withdrawal, codeine withdrawal, and morphine withdrawal.

17. The composition of claim 1, wherein the composition is administered through a capsule comprising a lipid.

18. A composition for treating opioid withdrawal, the composition comprising:
between about 2.5 to 100 milligrams of a cannabinoid, the cannabinoid being selected from the group consisting of: cannabinol, cannabidiol, cannabigerol, cannabichromene, and Delta-9 tetrahydrocannabinol, and mixtures thereof;
between about 0.084 to 33.6 milligrams of a terpenoid, or between about 0.3% to 0.6% of the composition, the terpenoid being selected from the group consisting of: a-Pinene, Mycerne, b-Caryophyllene, a-Bisabolol, Linalool, Limonene, Terpinolene, Eucalyptol, Terpineol, Camphene, and mixtures thereof;
between about 0.5 to 3000 milligrams of an herb, the herb being selected from the group consisting of: Tumeric, Curcumin, Bromelain, Pineapple, Capsaicin, Devil's Claw, Harpagoside, Ginseng, Ginsenosides, and mixtures thereof;
between about 25 to 2500 milligrams of a medicinal mushroom, the medicinal mushroom being selected from the group consisting of: Lion's Mane, Hericium erinaceus, Reishi, Ganoderma lucidum, Cordyceps, Turkey Tail, Trametes versicolor, Shiitake, Maitake, and mixtures thereof;
whereby the lipophilic nature of the terpenoid creates a medicinal entourage effect with the cannabinoid to enhance the bioavailability of the cannabinoid, so as to help inhibit symptoms of opioid withdrawal;
whereby the herb binds to and activates cannabinoid receptors in the body to enhance the pain relief capacity of the cannabinoid, so as to help inhibit symptoms of opioid withdrawal; and
whereby the medicinal mushroom comprises a psychedelic compound that produce a therapeutic effect to enhance the pain relief capacity of the cannabinoid, so as to help inhibit symptoms of opioid withdrawal.

19. The composition of claim 18, wherein the composition comprises a first set of ingredients for a first withdrawal phase embodiment, a second set of ingredients for a second withdrawal phase embodiment, and a third set of ingredients for a pain management embodiment.

20. A method for manufacturing a composition for treating opioid withdrawal, the method comprising:
soaking a cannabinoid, a terpenoid, an herb, and a medicinal mushroom in a liquid;
combining the cannabinoid, the terpenoid, the herb, and the medicinal mushroom;
decocting the combination of the cannabinoid, the terpenoid, the herb, and the medicinal mushroom;
combining, distilling, and evaporating the concentrated liquids to produce a dry powder;
forming the dry powder into a capsule;
encapsulating the capsule into a lipid-based panel; and
orally administering the lipid coated capsule.
